(19) United States
(12) Patent Application Publication
(10) Pub. No.: US 2004/0250729 A1
(43) Pub. Date: Dec. 16, 2004

(54) Fast-setting carbonated hydroxyapatite compositions and uses

(76) Inventors: Bor Z. Jang, Fargo, ND (US); Laixia Yang, Fargo, ND (US)

Correspondence Address: Bor Z Jang, 2902, 28 AVE, S.W., FARGO, ND, 58103, US

(21) Appl. No.: 10/460,869

(22) Filed: Jun. 16, 2003

(51) Int. Cl. A61F 2/28

(52) U.S. Cl. 106/35; 106/690; 106/691; 623/23.62

(57) ABSTRACT

A fast-setting two-part calcium phosphate cement formulation that, when mixed, is capable of hardening and forming an integral mass in less than four minutes. The integral mass is approximately 2 to 10 wt % carbonate-substituted hydroxyapatite that has a calcium/phosphate molar ratio of about 1.33 to 2.0. The cement formulation contains: (a) ultra-fine dry powder ingredients, with an average particle size smaller than 2 μm (preferably smaller than 0.5 μm and most preferably smaller than 100 nanometers) in diameter, comprising a partially neutralized phosphoric acid, a calcium phosphate source, and calcium carbonate in an amount ranging from about 9.33 to 70 wt % of the dry powder ingredients; and (b) a physiologically acceptable aqueous lubricant solution selected from the group consisting of 0.01 to 2M sodium phosphate solution at pH 6 to 11 and 0.01 to 2M sodium carbonate solution at pH 6 to 11, wherein the aqueous lubricant solution is present in an amount ranging from about 15 to 50 wt % of the two-part calcium phosphate cement formulation. The formulation can be used as a bone or teeth substitute or filler material.

### FIELD OF THE INVENTION

The present invention is related to the preparation of fast-setting compositions of carbonate-substituted forms of hydroxyapatite, and the biomedical use of such compositions.

### BACKGROUND OF THE INVENTION

Hydroxyapatite (HAp) materials are known to exhibit the basic properties of human bones and teeth. A considerable amount of research has been conducted on the remineralization of incipient dental lesions by deposition of hydroxyapatite, Ca₁₀(PO₄)₆ (OH)₂, on such lesions. Remineralization of tooth enamel has been carried out experimentally both in vivo and in vitro. These studies have focused on the remineralizing properties of saliva and synthetic solutions supersaturated with respect to HAp.

Calcium-based implants also have been used for the replacement of skeletal tissues. Most of these implants have been in the form of prefabricated, sintered HAp in either granule or block forms. These forms of HAp have several drawbacks: (a) a limited ability to conform to skeletal defects (particularly in the block form), (b) inadequate structural integrity of granules (which do not bond together), and (c) difficulty in molding the implant to the shape of missing skeletal tissue with both blocks and granules. The block form of HAp provides structural support, but must be held in place by mechanical means. This constraint greatly limits its use and its cosmetic results. It is very difficult to machine a shape from a brittle ceramic material such that it fits a particular defect. The granular form produces cosmetically better results, but has a very limited structural stability and is difficult to contain during and after a surgical procedure. In general, all of these products are ceramics, produced by high temperature sintering. These ceramic-type materials are in general functionally non-resorbable in a living body.

In addition to HAp, there are a number of other calcium phosphate minerals, such as fluorapatite, octacalcium phosphate, whitlockite, brushite and monetite, which are known to be relatively biocompatible. However, different crystalline forms of the calcium phosphate minerals exhibit different levels of resorbability; e.g., octacalcium phosphate and whitlockite are less resorbable than brushite or monetite in a living body.

Broadly speaking, apatite is a particularly interesting class of materials for biomedical applications. The term “apatite” refers to a wide range of compounds represented by the general formula M²⁺₁₀ (ZO₄³⁻)₆ Y⁻₂, where M is a metal atom (particularly an alkali or alkaline earth atom), ZO₄ is an acid radical, where Z may be phosphorous, arsenic, vanadium, sulphur, silicon, or may be substituted in whole or in part by carbonate (CO₃²⁻), and Y is an anion (usually halide, hydroxy, or carbonate). When ZO₄³⁻ is partially or wholly replaced by trivalent anions (such as CO₃²⁻) and/or Y⁻ is partially or wholly replaced by divalent anions, then charge balance may be maintained in the overall structure by the presence of additional monovalent cations (such as Na⁺) and/or protonated acid radicals (such as HPO₄²⁻).

Among the apatite group, hydroxyapatite (HAp) and its various derivatives or variants, have been recognized to be a major structural component of biological tissues (e.g., as indicated earlier, bone and teeth, and some invertebrate skeletons). In many clinical situations, it would be desirable to be able to replace or strengthen the bone structure. These include the situations of broken bone, surgically removed bone portions, destroyed bone, degraded bone, and brittle bone.

For use as a bone substitute, the material should ideally exhibit certain characteristics that facilitate the production, storage life, and biomedical application of the material. It is desirable to have a material which could be percutaneously injected as a flowable composition to fill in voids or areas deficient of hard bone. In the situations where the material is to be placed in the body and shaped and hardened in situ, one must consider the following: (a) the rate at which hydroxyapatite forms, (b) the extent to which the formation of hydroxyapatite is exothermic, (c) possible generation of gas as a by-product, and (d) physiological acceptance of the material at all phases of curing to the final product, so as to avoid the initiation of clotting, inflammatory responses, and other undesirable side effects. A highly exothermic reaction may possibly cause thermal necrosis of the surrounding tissue. In some situations a fast-setting composition is preferred due to the fact that the blood in an intended site could otherwise wash away or dissolve mineral salts prior to their setting up as an insoluble mineral. Furthermore, some blood components may become incorporated into the mineral, thereby significantly changing its physical and mechanical properties.

Two different forms of apatite are particularly useful. One form is an hydroxyapatite or its fluoridated derivative that is non-resorbable in vivo. The other includes forms of apatite that are substantially resorbable in vivo. For substitute bone applications, both forms of apatite must be strong and non-brittle. It is also desirable to have a strong adhesion between apatite and the remaining bone or calcified tissue. It is further desirable for apatite to perform other functions of natural bone such as (a) to accommodate stem cells; (b) to allow infiltration by cells normally resident in natural bone such as osteoclasts and osteoblasts; (c) to allow remodeling of the material by the infiltrating cells followed by new bone in-growth; and (d) to act in metabolic calcium exchange in a manner similar to native bone.

It is known in the field that carbonate plays an important role in dictating the processing, structure and properties of the resulting HAp. During the apatite forming process, the presence of carbonate in the HAp reactants tends to inhibit crystal growth of HAp, resulting in much smaller crystals with enhanced solubility of carbonated HAp, which is a desirable feature. Carbonates are known to occur in the apatites of hard tissues, and their presence changes the properties of stoichiometric apatite. In addition to causing a reduction in crystallite size, carbonates have been found to be capable of inducing (a) changes in the morphologies of the mineral phase from needles and rods to equi-axial crystals or spheroids, (b) contraction of the a-axis and an expansion in the c-axis, (c) internal strain, and (d) chemical instability. All of these features tend to result in higher solubilities of carbonate-substituted HAp. The structural changes as revealed by the x-ray diffraction patterns and the radial distribution function indicate that, as the concentration of carbonate increases, the patterns become more amorphous in character. The line broadening observed in the diffraction pattern is believed to be caused by decreasing crystallite size and degree of crystallinity. In addition to inhibiting HAp crystal growth, carbonate substitution significantly increases the solubility of HAp. Whether the carbonates are structurally bound within or absorbed onto HAp makes a difference in dissolution behavior. Dissolution was found to increase in HAps containing structurally bound carbonates, while it decreases in HAps with absorbed CO₃²⁻. The decrease in dissolution was presumably due to the hydronium ions having to compete for the surface of HAp, hence the deposition of the CO₃²⁻ layer was required.

The amount of carbonate being incorporated during HAp precipitation under normal physiological conditions is approximately 1% by weight CO₃²⁻, which is relatively low as compared to natural bone that consists of approximately 4% by weight CO₃²⁻. Bone mineral apatite with a level of carbonate between 2% and 10% by weight is commonly referred to as dahllite.

Carbonates can substitute in both the Z and Y sites of the apatite structure, M²⁺₁₀ (ZO₄³⁻)₆ Y⁻₂. Carbonates substituted for PO₄³⁻ groups during precipitation reactions tends to result in the formation of HAp, Ca₁₀(PO₄)₆(OH)₂. Specifically, the HAp formed at lower temperatures exhibits carbonate substitution at the phosphate sites. Due to the smaller size of carbonate as compared with phosphate, this substitution results in a decrease in the a-axis of the apatite. In contrast, crystallographically, in most apatites formed at higher temperatures, the carbonates are found in the vicinity of the six-fold axis, where they replace hydroxyl ions. Since the carbonate is larger than the hydroxyl ion, an increase in the a-axis results.

It is also known in the field of bio-materials that the skeleton in a body is the reservoir for almost all of the body's calcium and most of its phosphorus and magnesium. The carbonate levels in human enamel have been shown to increase in concentration from the surface to the dentin. The carbonate concentration in the surface enamel has also been shown to decrease with age. The ease of ionic substitution in the apatite lattice allows for the substitution of ions from the fluids surrounding the bone, and vice versa. This notion seems to suggest that hard tissues act as a regulatory reservoir for certain ions by incorporating ions into its structure when ionic concentration in the serum rises too high, and dissolving ions when the body is deficient in them. Possible candidates for this type of ions include some of the inorganic constituents of serum such as ionized and complexed calcium, inorganic phosphates, magnesium, bicarbonate, sodium, chloride, potassium, among others.

Carbonate appears to be required for the cellular infiltration of bone by osteoclasts, osteoblasts and other bone resident cells. Since osteoclasts and osteoblasts are involved in mineral replacement and bone remodeling, it would be advantageous to use a carbonated form of apatite, or dahllite, in any synthetic apatite-associated implant. Because dahllite can be remodeled by the body's natural processes, the dahllite component of an implant should eventually be replaced by natural bone, through the action of osteoclasts and osteoblasts. Thus, dahllite implants should eventually exhibit many or all of the desirable features of natural bone such as increased strength, elasticity and durability.

Prior-art methods of chemically forming hydroxyapatite have not produced modified hydroxyapatites with a significant level of structurally incorporated carbonate. This is primarily due to the acid present in the reactions of other methods tending to react with the carbonate to produce gaseous CO₂. The generation of gaseous by-products tend to produce bubble-containing apatite structures of compromised mechanical integrity. It is therefore desirable to develop a method for producing dahllite with a sufficient level of carbonate substitution in the apatite despite the presence of the acid required to form the apatitic structure. Although Constantz, et al. have disclosed a method and related formulation for producing dahllite (U.S. Pat. No. 5,900,254, May 4, 1999 and U.S. Pat. No. 5,336,264, Aug. 9, 1999), the method and formulation do not provide a composition that sets and hardens in less than 4 minutes. In many clinical situations, it is advantageous to have the desired apatite structure as fast as possible, provided that fast setting does not lead to reduced strength of the resulting structure or make it difficult to complete the clinical procedures (e.g., paste injection into the intended body site).

The following relevant U.S. patents are representative of the state-of-the-art for the field of hydroxyapatite, carbonated hydroxyapatite, and their derivatives or variants:

1. R. O'Leary et al., “Flowable Demineralized Bone Powder Composition and Its Use in Bone Repair”, U.S. Pat. No. 5,073,373 (Dec. 17, 1991).

2. I. Ison et al., “Storage Stable Calcium Phosphate Cements”, U.S. Pat. No. 6,053,970 (Apr. 25, 2000).

3. M. Sumita, “Composition for Forming Calcium Phosphate Type Setting Material and Process for Producing Setting Material”, U.S. Pat. No. 5,281,404 (Jan. 25, 1994).

4. L. Chow, “Calcium Phosphate Hydroxyapatite Precursor and Methods for Making and Using the Same”, U.S. Pat. No. 5,695,729 (Dec. 9, 1997).

5. W. Brown et al., “Combinations of Sparingly Soluble Calcium Phosphates in Slurries and Pastes as Mineralizers and Cements”, U.S. Pat. No. Re. 33,161 (Feb. 6, 1990).

6. W. Brown et al., “Dental Restorative Cement Pastes”, U.S. Pat. No. Re. 33,221 (May 22, 1990).

7. L. Chow et al., “Calcium Phosphate Hydroxyapatite Precursor and Methods for Making and Using the Same”, U.S. Pat. No. 5,522,893 (Jun. 4, 1996).

8. L. Chow et al., “Self-Setting Calcium Phosphate Cements and Methods for Preparing and Using Them”, U.S. Pat. No. 5,525,148 (Jun. 11, 1996).

9. L. Chow et al., “Calcium Phosphate Hydroxyapatite Precursor and Methods for Making and Using the Same”, U.S. Pat. No. 5,545,254 (Aug. 13, 1996).

10. L. Chow et al., “Calcium Phosphate Hydroxyapatite Precursor and Methods for Making and Using the Same”, U.S. Pat. No. 6,325,992 B1 (Dec. 4, 2001).

11. B. Constantz, “In Situ Calcium Phosphate Minerals Method”, U.S. Pat. No. 4,047,031 (Sep. 10, 1991).

12. B. Constantz, et al., “Intimate Mixture of Calcium and Phosphate Sources as Precursor to Hydroxyapatite”, U.S. Pat. No. 5,053,212 (Oct. 1, 1991).

13. B. Constantz, “Methods for In Situ Prepared Calcium Phosphate Minerals”, U.S. Pat. No. 5,129,905 (Jul. 14, 1992).

14. B. Constantz, et al., “Situ Prepared Calcium Phosphate Composition and Method”, U.S. Pat. No. 5,336,264 (Aug. 9, 1994).

15. B. Constantz, “Carbonated Hydroxyapatite Compositions and Uses”, U.S. Pat. No. 5,900,254 (May 4, 1999).

16. B. Constantz, “Paste Compositions Capable of Setting into Carbonated Apatite”, U.S. Pat. No. 5,952,010 (Sep. 14, 1999).

17. B. Constantz, “Carbonated Hydroxyapatite Compositions and Uses”, U.S. Pat. No. 5,962,028 (Oct. 5, 1999).

18. B. Constantz et al., “Kits for Preparing Calcium Phosphate Minerals”, U.S. Pat. No. 6,002,065 (Dec. 14, 1999).

19. B. Constantz, “Paste Compositions Capable of Setting into Carbonated Apatite”, U.S. Pat. No. 6,334,891 (Jan. 1, 2002).

20. P. Brown, “Bone Substitute Composition Comprising Hydroxyapatite and a Method of Production Therefor”, U.S. Pat. No. 6,201,039 (Mar. 13, 2001).

21. H. Yamazaki et al., “Method of Manufacturing Hydroxyapatite and Aqueous Solution of Biocompounds at the Same Time”, U.S. Pat. No. 6,149,796 (Nov. 21, 2000).

22. U. Ripamonti et al., “Biomaterial and Bone Implant for Bone Repair and Replacement”, U.S. Pat. No. 6,302,913 (Oct. 16, 2001).

23. K. Marra et al., “Biocompatible Compositions and Methods of Using Same”, U.S. Pat. No. 6,165,486 (Dec. 26, 2000).

24. D. Lee et al., “Bone Substitution Material and a Method of Its Manufacture”, U.S. Pat. No. 6,214,368 B1 (Apr. 10, 2001).

25. F. H. Lin et al., “α-TCP/HAP Biphasic Cement and Its Preparing Process”, U.S. Pat. No. 6,338,752 B1 (Jan. 15, 2002).

26. J. Carpena et al., “Method for Making Apatite Ceramics, In Particular for Biological Use”, U.S. Pat. No. 6,338,810 (Jan. 15, 2002).

27. M. Akashi et al., “Hydroxyapatite, Composite, Processes for Producing These, and Use of These”, U.S. Pat. No. 6,395,037 B1 (May 28, 2002).

28. B. Edwards et al., “Porous Calcium Phosphate Cement”, U.S. Pat. No. 6,547,866 B1 (Apr. 15, 2003).

29. P. Higham, “Calcium Phosphate Composition and Method of Preparing Same”, U.S. Pat. No. 6,558,709 B2 (May 6, 2003).

30. A. Gertzman et al., “Malleable Paste for Filling Bone Defects”, U.S. Pat. No. 6,030,635 (Feb. 29, 2000).

31. F. Dorigatti et al., “Biomaterials for Bone Replacements”, U.S. Pat. No. 6,533,820 B2 (Mar. 18, 2003).

32. S. T. Liu et al., “Resorbable Bioactive Phosphate Containing Cements”, U.S. Pat. No. 5,262,166 (Nov. 16, 1993).

33. Y. Hakamatsuka et al., “Method of Preparing Calcium Phosphate”, U.S. Pat. No. 5,322,675 (Jun. 21, 1994).

34. M. Hirano et al., “Calcium Phosphate Granular Cement and Method for Producing Same”, U.S. Pat. No. 5,338,356 (Aug. 16, 1994).

35. A. Imura et al., “Tetracalcium Phosphate-Based Materials and Processes for Their Preparation”, U.S. Pat. No. 5,536,575 (Jul. 16, 1996).

36. M. Fulmer et al., “Reactive Tricalcium Phosphate Compositions”, U.S. Pat. No. 5,709,742 (Jan. 20, 1998).

### SUMMARY OF THE INVENTION

The present invention provides compositions that are comprised of dahllite, analogs thereof, or otherwise carbonate-substituted forms of hydroxyapatite (dahllite-like compositions). These compositions are useful in a variety of biomedical applications. The compositions can be prepared in two parts, one in a dry powder state and the other in a wet fluid state. The powder particles should preferably have an average particle size of two (2) μm or smaller, more preferably 0.5 μm or smaller, and most preferably 0.1 μm (or 100 nm) or smaller. The two parts can be mixed together to become a mixture that is flowable, moldable, and capable of hardening in situ in a patient's body. The compositions harden, normally in less than four minutes and preferably in less than two minutes, into polycrystalline structures that, if so desired, can be shaped subsequent to hardening.

### DESCRIPTION OF PREFERRED EMBODIMENTS

The present invention provides carbonated hydroxyapatite compositions commonly referred to as dahllite-like materials. The compositions can be used to substitute many of the functions of naturally occurring calcified tissues or to repair such tissues as teeth and bone.

A preferred embodiment of the present invention is a two-part calcium phosphate cement formulation that, when mixed, is capable of hardening and forming an integral mass, which is approximately 2 to 10 wt % carbonate-substituted hydroxyapatite that has a calcium/phosphate molar ratio of about 1.33 to 2.0. The two-part calcium phosphate cement formulation contains a dry powder part and a wet fluid part. The powder part comprises ultra-fine dry powder particles, with an average particle size smaller than 2 μm in diameter. The powders include primarily a partially neutralized phosphoric acid, a calcium phosphate source, and calcium carbonate in an amount ranging front about 9.33 to 70 wt % of the dry powder part. The wet fluid part contains a physiologically acceptable aqueous lubricant solution, which is either a 0.01 to 2M sodium phosphate solution at pH 6 to 11 or a 0.01 to 2M sodium carbonate solution at pH 6 to 11. The aqueous lubricant solution is present in an amount ranging from about 15 to 50 wt % of the two-part calcium phosphate cement formulation.

The carbonated HAp or dahllite-like products can be readily formed by combining the wet and dry parts to provide a substantially uniform mixture, shaping the mixture into desired dimensions, and allowing the mixture to harden. During hardening (or setting), the mixture crystallizes into a solid apatite structure. Alternatively, the dahllite-like apatitic compositions can also be shaped after hardening is complete. For bone repair or substitution, the dahllite-like apatitic compositions can be in the form of precursor reaction mixtures that are placed (e.g., via syringe injection) into an intended defect site of a patient's body and hardened in situ.

The preferred powder particle sizes are 2 μm or smaller. The further preferred average particle sizes are 0.5 μm or smaller and most preferred average particle sizes are 0.1 μm (100 nm) or smaller. With average particle sizes being smaller than 2 μm, the setting time of the dry and wet parts when mixed together is typically four (4) minutes or shorter at a setting temperature of 37° C. in air. The setting time is reduced to approximately two (2) minutes or shorter when the mixture is made from finer particles with an average particle size smaller than 0.5 μm. Still finer particles (100 nm or smaller) only lead to a slightly shorter setting time (less than 2 minutes), but result in improved mechanical properties of the carbonated HAp.

The composition of the carbonated hydroxyapatite may vary. For instance, the calcium/phosphate ratio may vary from 1.33 to 2.0 with 1.67 being the natural ratio. With the ratio smaller than 1.67, there will be a defective lattice structure from the calcium vacancies. For a ratio of 1.33, there will be two calcium ions absent. The extra hydrogens may be up to about 2 hydrogen ions per phosphate, usually not more than about one hydrogen ion per phosphate. The ions will be uniformly distributed throughout the product.

The dry powder reactant typically consists of a phosphoric acid source substantially free of unbound water, an alkali earth metal source (particularly calcium source), optionally crystalline nuclei (particularly hydroxyapatite or calcium phosphate crystals), and calcium carbonate. The wet fluid part or reactant typically comprises a physiologically acceptable lubricant (e.g., water), which may contain various solutes. The dry ingredients may be prepared as a mixture of ultra-fine powders and subsequently combined with the liquid ingredients.

Specifically, the phosphoric acid source may be any partially neutralized phosphoric acid, particularly up to complete neutralization of the first proton as in calcium phosphate monobasic. It can consist of orthophosphoric acid, possibly in a crystalline form, which is substantially free of combined water. The acid source will generally be about 15 to 35 weight percent of the dry components of the mixture, more usually 15 to 25 weight percent.

The calcium source could play a dual role of providing calcium and acting as a neutralizing agent. The desired final product depends on the relative ratios of calcium and phosphate. Calcium sources generally include counter-ions such as carbonate and phosphate. Dual sources of calcium and phosphate such as tetra-calcium phosphate or tri-calcium phosphate are particularly useful. The proportion of tetra-calcium phosphate or tri-calcium phosphate in the mixture may typically lie from about 0 to 70 weight percent, more preferably from about 0 to 40 weight percent, and most preferably from about 2 to 18 weight percent of dry weight of the dry components of the mixture.

One major advantage of having calcium carbonate being present to serve as a source of calcium and carbonate is that it also serves to neutralize the acid and, hence, the reaction results in relatively little temperature rise. However, there is substantial evolution of gas which must be released during mixing. Calcium carbonate will be present in the mixture from about 2 to 70 weight percent, preferably from about 2 to 40 weight percent, and most preferably from about 2 to 18 weight percent of dry weight of the dry components of the mixture. Calcium hydroxide may also be present in the mixture from about 0 to 40 wt. %., preferably from about 2 to 25 wt. %, and most preferably from about 2 to 20 wt. %.

Halides such as fluorine and chlorine may be added to form fluorapatite (francolite), or chlorapatite, respectively. The sources of fluoride or chloride will include either soluble salts such as calcium chloride, calcium hexafluorosilicate or sodium fluoride. The source may be added as a dilute acid in the aqueous lubricant, generally at concentrations of less then about 1M. Halides could constitute from about 0 to 4 weight percent, more usually from about 2 to 4 weight percent, and preferably from about 3 to 4 weight percent of dry weight. Usually at least about 5%, more usually at least about 10% of the hydroxyl groups will be replaced. Francolite may potentially find applications in dentistry.

Preferably all the dry powder components are combined to form the dry powder part of the two-part composition. Alternatively, one may choose to dissolve a small amount of a dry powder ingredient in the wet lubricant part to adjust the consistency of the wet fluid part of the two-part composition. This could also help to improve the uniformity of the various components, dry and wet, when combined together to form a reactive mass. Various solutes may be included in the wet fluid part. For instance, a gel or colloid, which has as a solute alkali metal hydroxide, acetate, phosphate, or carbonate, particularly sodium, more particularly phosphate or carbonate, may be added at a concentration in the range of 0.01 to 2 M, particularly 0.05 to 0.5 M, and at a pH in the range of about 6-11, more usually about 7-9, particularly 7-7.5.

Various dry powders may be size-reduced to 2 μm (or preferably 0.5 μm and further preferably 100 nm) or smaller via ball milling. The high-energy planetary ball mill available from Nanotek Instruments, Inc. (Fargo, N.D.) is capable of reducing various ceramic powders down to nanometer scales. The dry components may be ball-milled separately and then combined to form a mixture or, alternatively, are combined to form a mixture of dry powders, which are then ball-milled to the desired size scales. The particle sizes, to a great extent, dictate the setting time of the resulting mixture of dry and wet components.

By varying the proportion of liquid lubricant, particularly water, added to the subject mixtures, the fluidity of the composition can be adjusted. Other water soluble and compatible liquids that are pharmacologically acceptable may be added to the wet fluid part of the two-part composition. These may include alkanols, more particularly polyols, such as ethylene glycol, propylene glycol or glycerol. These diluents or thickening agents may be present in less than about 10 volume percent in an appropriate medium. The liquid will generally be from about 15 to 50, more usually from about 20 to 35. weight percent of the entire composition, dry and wet components together.

For bone repair or substitution applications, implantation of the mixture of dry and wet components may be by syringe or catheter injection. The composition may be used as a paste that passes through a needle in the range of about 10-18 gauge, preferably about 14-16 gauge. Nanometer-scaled particles appear to make syringe injection a lot easier, also with a reduced risk of clogging up the needle. If less lubricant is added, the composition is kneadable or moldable, being capable of forming clay-like putty that may be molded prior to setting. The setting time of the compositions can be varied, to a great extent, by varying the solid powder particle size and, to a lesser extent, by changing the liquid proportion.

After mixing, the invented compositions will undergo chemical reactions to become hardened. During hardening, crystal growth occurs and the product becomes an integral mass. The resulting mass will have a composition that contains structurally incorporated carbonate in the apatite structure. The carbonate proportion lies between about 2% and about 10% carbonate by weight, usually between 2.5% to 7%, and optimally between about 4% to about 6% carbonate by weight.

The un-cured compositions could have a pH in the range of about 5.5-8.5, but usually in the range of about 6-7.5. They can be administered to an environment having a temperature in the range of about 0-45° C., usually 20-40° C., and optimally about normal physiological temperature, 37° C. The compositions are bio-compatible, having low or no toxicity when prepared in accordance with described methods. They are readily resorbable in vivo and, hence, the set mass could be gradually replaced by natural bone.

For some clinical applications, it may be advantageous to include additional components into the mixture during the formation of the carbonated hydroxyapatite. Examples of useful components are pharmacologically active agents, proteins, polysaccharides, and other biocompatible polymers. Of particular utilization value are proteins involved in skeletal structure such as various forms of collagen (fibrin, fibrinogen, keratin, tubulin, elastin, etc.) or structural polysaccharides, such as chitin. Pharmacologically active agents that might be added include drugs that enhance bone growth, serve as a variety of cell growth factors, or act as anti-inflammatory or anti-microbial agents. Examples of such agents include bone morphogenetic protein (BMP), cartilage induction factor, platelet derived growth factor, and skeletal growth factor.

Pharmacologically active agents or structural proteins may be added as an aqueous dispersion or solution. The protein usually will be present in from about 1-10 wt % of the aqueous dispersion. After hardening, the resulting composition will contain the protein in from about 0.01 to 10, usually from about 0.05 to 5 weight percent. By varying the proportions of the reactants, one can obtain compositions with varying and predictable rates of resorption in vivo. In sum, a clinician can add drug and inorganic components to the invented compositions in order to practice an implantable or injectable time-release delivery platform for drugs, inorganic mineral supplements, or the like.

For use as cements or fillers for bone or tooth repair applications, the invented compositions are capable of bonding to other apatites in existing bones or teeth, which are mainly composed of dahllite and collagen. The compositions strongly adhere to surfaces that are wet or coated with saliva, blood or lymphatic fluid. They are capable of filling in voids, conforming to irregular surfaces such as concavities and convexities, and providing for the structural functions of replaced connective tissue.

The invented compositions can be used to form carbonated hydroxyapatite coatings on implants or other formed objects. The composition, as a flowable or formable product, can serve as a bone cement, or an infiltrate cement for the treatment of osteoporotic bone. Paste or clay-like mixtures may be formed and hardened into a carbonated hydroxyapatite product, either externally or in situ.

One specifically preferred embodiment of the present invention is the preparation of carbonated hydroxyapatite by a process whereby a calcium source (at least one component of which is calcium carbonate) and an acidic phosphate source (optionally comprised of orthophosphoric acid crystals substantially free of uncombined water) are mechanically mixed for sufficient time for a partial reaction between the calcium source and acidic phosphate source to occur. The partially reacted composition, in the form of a fine powder with average particle size smaller than 2 μm (preferably smaller than 0.5 μm and most preferably smaller than 100 nm), can be subsequently mixed with a physiologically suitable lubricant fluid which varies the fluidity of the product and allows for substantially complete reaction of the reactants. The final mixture may be subsequently shaped and hardened, hardened then shaped, or placed in the body and hardened in situ, eventually resulting in a solid carbonated hydroxyapatite product. The resulting carbonated hydroxyapatite will have substantially reduced reaction heat or non-exothermic setting. This low- or non-exothermal reaction is advantageous because it provides for the stability of introduced pharmacological agents, and, when hardened in situ, provides a reduced level of patient discomfort. The compositions prepared in this manner are also applicable for use as bone cements or fillers, dental or endodontic filling agents, coatings for implantable substrates, or formed into suitable shapes before or after hardening into a structure.

The calcium source used in the above process will typically include a mixture of tetra-calcium phosphate (TCP) and calcium carbonate with the former typically present in from about 55 to 75 wt. %, or more usually 60-70 wt. %, and the latter typically present in from about 1 to 40 wt. %, or more typically 2 to 18 wt. % of the dry weight of the total reaction mixture. The acid phosphate source will be about 15 to 35, or more preferably 15 to 25 wt. % of the dry weight of the reaction mixture.

Alternatively, the composition may typically include a mixture of tri-calcium phosphate (TrCP), calcium carbonate (CC), and calcium hydroxide (CH) with TrCP typically present in from about 50 to 90 wt. %, or more usually 75 to 90 wt. %, CC typically present from about 1 to 40 wt. % or more usually 2 to 18 wt. %, and CH typically present from about 0 to 40 wt. % or more-usually 2 to 20 wt. % of the dry weight of the total reaction mixture. The acid phosphate source for this mixture will be about 5 to 35 wt. % or more usually 5 to 25 wt. % of the dry weight of the reaction mixture. A fluoride source may be added to the mixture in an amount from about 0 to 4 wt. %, preferably 3 to 4 wt. % of dry weight.

After the dry ingredients are combined, the reactants will be placed in intimate contact by ball milling for the purposes of reducing the particle sizes and facilitating partial reactions between selected ingredients, if so desired. The product that has undergone a partial reaction will require less lubricant to provide a workable mixture and will result in a reduced setting time of the final mixture.

The invented compositions may be prepared in the form of a kit that comprises two components, one being dry powder component and the other wet fluid lubricant component. This form is particularly convenient for use in a clinical situation that requires bone repair or substitution.

### EXAMPLE 1

Five samples, A-E, were prepared. In each sample, a mixture of dry powders (tetra-calcium phosphate, calcium carbonate, and orthophosphoric acid) was prepared by using a high-energy plenary ball mill to reduce the particle sizes to a desired average value. The dry powder ingredients were then combined with a desired amount of sodium phosphate solution, with the setting time of the resulting mixture measured. Individual samples were analyzed by Fourier transform infrared spectroscopy (FTIR) using pressed KBr pellets, by carbon coulometry using acidification for total inorganic carbon analysis, and by combustion for total carbon analysis. The samples were further assayed for carbonate content in duplicate. The results of these studies are presented in Table 1, which indicates that the setting time decreases with decreasing powder particle sizes. The formation time the carbonated hydroxyapatite can be reduced to below 2 minutes if the powder particles are nanometer-scaled.

TABLE 1

| | | Average | | Setting |
|---|---|---|---|---|
| Sample | Formulation | Particle Size | % Carbonate | Time |
| A | 23 g tetra-calcium phosphate (TCP) | 8.5 μm | 4.53 | 55 min |
| | 2.8 g calcium carbonate (CC) | | | |
| | 4.12 g orthophosphoric acid (OPA) | | | |
| | Wet fluid part: | | | |
| | 15 g of 0.1 M sodium phosphate (SP) solution | | | |
| B | Same as in Sample A | 4.5 μm | 4.55 | 8 min |
| C | Same as in Sample A | 2.3 μm | 4.56 | 3 min |
| D | Same as in Sample A | 0.45 μm | 4.58 | 2 min |
| E | Same as in Sample A | 80 nm | 4.58 | <2 min |

1. A two-part calcium phosphate cement formulation that, when mixed, is capable of hardening and forming an integral mass, wherein said integral mass is approximately 2 to 10 wt % carbonate-substituted hydroxyapatite that has a calcium/phosphate molar ratio of about 1.33 to 2.0, said cement formulation comprising:
(A) as the first part, ultra-fine dry powder ingredients, with an average particle size smaller than 2 μm in diameter, comprising a partially neutralized phosphoric acid, a calcium phosphate source, and calcium carbonate in an amount ranging from about 9.33 to 70 wt % of said dry powder ingredients; and
(B) as the second part, a physiologically acceptable aqueous lubricant solution selected from the group consisting of 0.01 to 2M sodium phosphate solution at pH 6 to 11 and 0.01 to 2M sodium carbonate solution at pH 6 to 11, wherein said aqueous lubricant solution is present in an amount ranging from about 15 to 50 wt % of the two-part calcium phosphate cement formulation.

2. The cement according to claim 1, wherein said average particle size is smaller than 0.5 μm.

3. The cement according to claim 1, wherein said average particle size is smaller than 100 nanometers.

4. The cement according to claim 1, 2, or 3, wherein said partially neutralized phosphoric acid source is Ca(H₂PO₄)₂H₂O.

5. The cement according to claim 1, 2, or 3, wherein said calcium phosphate source is tri-calcium phosphate.

6. A two-part calcium phosphate cement formulation that, when mixed, is capable of hardening and forming an integral mass in less than 4 minutes, wherein said integral mass is approximately 2 to 10 wt % carbonate-substituted hydroxyapatite that has a calcium/phosphate molar ratio of about 1.33 to 2.0 and is bio-compatible, said two-part calcium phosphate cement formulation comprising:
(A) as the first part, ultra-fine dry powder ingredients, with an average particle size less than 2 μm in diameter, comprising a partially neutralized phosphoric acid, a tri-calcium phosphate, and calcium carbonate in an amount ranging from about 9.33 to 40 wt % of said dry powder ingredients; and
(B) as the second part, a physiologically acceptable aqueous lubricant solution selected from the group consisting of 0.01 to 2M sodium phosphate solution at pH 6 to 11 and 0.01 to 2M sodium carbonate solution at pH 6 to 11, wherein said aqueous lubricant solution is present in an amount ranging from about 15 to 50 wt % of the two-part calcium phosphate cement formulation.

7. The cement according to claim 6, wherein said average particle size is smaller than 0.5 μm.

8. The cement according to claim 6, wherein said average particle size is smaller than 100 nanometers.

9. The cement according to claim 6, 7, or 8, wherein said aqueous lubricant solution is a 0.01 to 2M sodium phosphate solution at pH 6 to 11.

10. The cement according to claim 6, 7, or 8, wherein said partially neutralized phosphoric acid is Ca(H₂PO₄)₂H₂O.

11. A two-part calcium phosphate cement formulation that, when mixed, is capable of hardening and forming an integral mass in less than 4 minutes, wherein said integral mass is approximately 2 to 10 wt % carbonate-substituted hydroxyapatite that has a calcium/phosphate molar ratio of about 1.33 to 2.0 and is bio-compatible, said two-part calcium phosphate cement formulation comprising:
(A) as the first part, ultra-fine dry powder ingredients, with an average particle size smaller than 2 μm in diameter, comprising Ca(H₂PO₄)₂H₂O, a tri-calcium phosphate, and calcium carbonate in an amount ranging from about 9.33 to 18 wt % of said dry powder ingredients; and
(B) as the second part a 0.01 to 2M sodium phosphate solution at pH 6 to 11 present in an amount ranging from about 15 to 50 wt % of the two-part calcium phosphate cement formulation.

12. The cement according to claim 11, wherein said average particle sizes is smaller than 0.5 μm.

13. The cement according to claim 11, wherein said average particle sizes is smaller than 100 nanometers.

14. A kit for use in the preparation of a composition capable of hardening and forming air integral mass, wherein said integral mass is approximately 2 to 10 wt % carbonate-substituted hydroxyapatite that has a calcium/phosphate molar ratio of about 1.33 to 2.0 and is bio-compatible, said kit comprising:
(A) ultra-fine dry powder ingredients, with an average powder particle size smaller than 2 μm in diameter, comprising a partially neutralized phosphoric acid, a calcium phosphate source, and calcium carbonate in an amount ranging from about 9.33 to 70 wt % of said dry powder ingredients; and
(B) a physiologically acceptable aqueous lubricant solution selected from the group consisting of 0.01 to 2M sodium phosphate solution at pH 6 to 11 and 0.01 to 2M sodium carbonate solution at pH 6 to 11, wherein said aqueous lubricant solution is physically separated from the dry powder ingredients of said kit and is present in an amount ranging from about 15 to 50 wt % of the total weight of said dry powder ingredients and said aqueous lubricant of said kit.

15. The cement according to claim 14, wherein said average particle size is smaller than 0.5 μm.

16. The cement according to claim 14, wherein said average particle size is smaller than 100 nanometers.

17. The kit according to claim 14, wherein said partially neutralized phosphoric acid is Ca(H₂PO₄)₂H₂O.

18. The kit according to claim 14, wherein said calcium phosphate source is tri-calcium phosphate.

19. The kit according to claim 14, wherein said aqueous lubricant solution is a 0.01 to 2M sodium phosphate solution at pH 6 to 11.

20. The kit according to claim 14, wherein at least two of said dry ingredients are combined.

21. A kit for use in the preparation of a composition capable of hardening and forming an integral mass, wherein said integral mass is approximately 2 to 10 wt % carbonate-substituted hydroxyapatite that has a calcium/phosphate molar ratio of about 1.33 to 2.0 and is bio-compatible, said kit comprising:
(A) ultra-fine dry powder ingredients, with an average particle size smaller than 4 μm in diameter, comprising Ca(H₂PO₄)₂H₂O, a tri-calcium phosphate, and calcium carbonate in an amount ranging from about 9.33 to 40 wt % of said dry powder ingredients; and
(B) a 0.01 to 2M sodium phosphate solution at pH 6 to 11, wherein said sodium phosphate solution is physically separated from the dry powder ingredients of said kit and is present in an amount ranging from about 15 to 50 wt % of the total weight of said dry powder ingredients and said sodium phosphate solution of said kit.

22. The cement according to claim 21, wherein said average particle size is smaller than 0.5 μm.

23. The cement according to claim 21, wherein said average particle size is smaller than 100 nanometers.

24. A kit for use in the preparation of a composition capable of hardening and forming an integral mass, wherein said integral mass is approximately 2 to 10 wt % carbonate-substituted hydroxyapatite that has a calcium/phosphate molar ratio of about 1.33 to 2.0 and is bio-compatible, said kit comprising:
(A) ultra-fine dry powder ingredients, with an average powder particle size smaller than 2 μm in diameter, comprising Ca(H₂PO₄)₂H₂O, a tri-calcium phosphate, and calcium carbonate in an amount ranging from about 9.33 to 18 wt % of said dry powder ingredients; and
(B) a 0.01 to 2M sodium phosphate solution at pH 6 to 11, wherein said sodium phosphate solution is physically separated from the dry powder ingredients of said kit and is present in an amount ranging from about 15 to 50 wt % of the total weight of said dry powder ingredients and said sodium phosphate solution of said kit.

25. The cement according to claim 24, wherein said average particle size is smaller than 0.5 μm.

26. The cement according to claim 24, wherein said average particle size is smaller than 100 nanometers.
